SEQUENCE LISTING (1 sequence)

SEQ ID NO: 1 - DNA, 413 bp, synthetic construct
  source 1..413: mol_type = other DNA
tacgtgacttgctcccagttatgtgtgacagagcaggatttattcaagatactagccttatcctctatgaggaagttaaaccgaatttaacagagagaattcaggactatgacgtgtctcttgataaagcccttgatgaactaatggatggtgacatcatagtatttcagaaggatgaccctgaaaatgataacagtgaattacccaccgcaaaggagtatttccgagatctctaccaccgcgttgatgtcattttctgtgataaaacaatccctaatgatcctggatttgtggttacgttatcaaatagaatgaattattttcaggttgcaaagacagttgcacagaggctcaacacagatccaatgttgctgcagtttttcaagtctcaaggttatagggatggcccaggt

(19) United States
(12) Patent Application Publication
(10) Pub. No.: US 2024/0009182 A1
(43) Pub. Date: Jan. 11, 2024

(54) REPURPOSED COMPOUND AS THERAPEUTIC FOR HPV-ASSOCIATED CANCERS

(71) Applicant: The Chinese University of Hong Kong, Hong Kong (CN)

(72) Inventors: Kay Sheung, Paul CHAN, Hong Kong (CN); Siaw Shi BOON, Hong Kong (CN); Chichao XIA, Hong Kong (CN)

(21) Appl. No.: 18/333,872

(22) Filed: Jun. 13, 2023

Related U.S. Application Data
(60) Provisional application No. 63/366,377, filed on Jun. 14, 2022.

(51) Int. Cl. A61K 31/473; A61P 35/00; A61K 31/7088

(52) U.S. Cl. CPC A61K 31/473; A61P 35/00; A61K 31/7088

(57) ABSTRACT

The subject invention pertains to a composition comprising a deubiquitinase inhibitor, including a ubiquitin-specific protease 7-specific inhibitor, and methods of using said composition to treat cancer in subject. The ubiquitin-specific protease 7-specific inhibitor can be HBX 19818 and/or an siRNA, which can inhibit the proliferation of cancerous cells, specifically cancerous cells derived from an HPV infection. The methods can further reduce the growth of cancerous cells in an anchorage-independent manner, reduce cancer cell migration, and reduce cancer cell invasion through the basal membrane.

### CROSS-REFERENCE TO RELATED APPLICATION

This application claims the benefit of U.S. Provisional Patent Application Ser. No. 63/366,377, filed Jun. 14, 2022, which is hereby incorporated by reference in its entirety including any tables, figures, or drawings.

### REFERENCE TO SEQUENCE LISTING

The Sequence Listing for this application is labeled “CUHK.190XC1.xml” which was created on Jun. 7, 2023 and is 2,212 bytes. The entire content of the sequence listing is incorporated herein by reference in its entirety.

### BACKGROUND OF THE INVENTION

Human papillomavirus (HPV) is a major etiological agent contributing to 5% of all human cancers, and 7.5% of cancer deaths in women worldwide. Recent data showed that at least 500 new cervical cancer cases occurred annually in Hong Kong (see worldwide website: chp.gov.hk/en/healthtopics/contenU25/56.html), and 570,000 individuals were affected by cervical cancers globally (see Arbyn M, Weiderpass E, Bruni L, de Sanjose S, Saraiya M, Ferlay J, Bray F. Estimates of incidence and mortality of cervical cancer in 2018: a worldwide analysis. Lancet Glob Health. 2020 February; 8(2):e191-e203. doi: 10.1016/52214-109X(19)30482-6. Epub 2019 Dec. 4. Erratum in: Lancet Glob Health. 2022 January; 10(1):e41.). In the United States alone, it was estimated that at least 35,900 new cases of HPV-attributable cancers occurred annually, affecting both men and women (see worldwide website: cdc.gov/cancer/hpv/statistics/cases.htm). The incidence of HPV-associated cancers, particularly cervical cancer, may appear to reach a plateau, but this does not reflect the real scenario. When categorizing the HPV-associated cancers by the economic status of a region, a recent study showed that the incidence of HPV-associated anal and vulvar cancers is on the rise in low-income counties and countries. Moreover, there is an alarming trend of increase in the number of HPV-associated head and neck cancers. These figures may not decrease in the next decade.

An HPV infection can cause cancers in the uterine, cervix, oropharynx, tonsils, base of tongue, anus, rectum, vulva, vagina, and penis. The pre-malignant and early stage of HPV-associated cancers is potentially treatable; whereas, advanced cancers have a poor prognosis. To date, a therapeutic tailored for HPV-associated cancers is still not available. The ability of HPV to promote cancer formation is through the expression of both viral E6 and E7 oncoproteins. The proteins collaboratively disrupt normal cell cycle checkpoints, resist apoptosis, and accelerate the functions of host oncoproteins.

Currently, treatment for HPV-associated cancers relies on a standard therapy applicable for cancers in general, which are nonspecific, non-cell selective, and exert broad-spectrum killing or inhibitory effects. Lack of a targeted therapy is the current bottleneck in reducing morbidity and mortality of patients with HPV-associated cancers, as well as in reducing the treatment costs of individuals and society.

Therefore, there is a continuing need for a precise medical intervention to reduce the mortality of HPV-associated cancers, particularly in low-income countries where the disease burden is rising.

### BRIEF SUMMARY OF THE INVENTION

The subject invention pertains to compositions comprising a deubiquitinase inhibitor, preferably a ubiquitin-specific protease 7 (USP7)-specific inhibitor, and methods of using said compositions. In preferred embodiments, the USP7-specific inhibitor in HBX 19818. In certain embodiments, the compositions can exert a selective inhibitory effect on HPV-positive cancer cells, while leaving HPV-null cancer cells unaffected. In certain embodiments, HPV E7 can form a complex with USP7, which is a deubiquitinating enzyme of host cells. This binding brings about an increased E7 steady-state level. After a treatment with the present compositions, subjects can have a decreased E7 protein steady-state level, as well as greatly reducing the ability of HPV-containing cancer cells to proliferate, grow in an anchorage-independent manner, migrate, and invade through the basal membrane. In certain embodiments, the compositions can suppress the oncogenic phenotype of HPV-associated cancer cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F HBX 19818 treatment suppressed the HPV16 E7 protein level. FIG. 1A. The representative immunoblots showing the level of HPV16 E7 constitutively expressed in CaSki cells upon treatment with inhibitors specific for USP7 (P5091, FT671, GNE-6776 and HBX 19818) and that targeting pan-USPs (PR-619) at the indicated concentrations. DMSO served as a vehicle control treatment. Cells were then collected and lysed, the levels of USP7 and HPV16 E7 proteins were detected using the respective specific antibodies. Beta-actin (β-actin) was included as a loading control. KDa: kilodalton. FIG. 1B. The bar chart showing the relative protein level of HPV16 E7 normalized by β-actin (n=3). Data are expressed as mean±standard error of the mean (SEM); Right panel showing the images at 100× magnification of the morphology of the CaSki cells treated with P5091, FT671, GNE-6776, HBX 19818 and PR-619 at concentrations indicated. FIG. 1C. The immunoblots showing the levels of USP7 and HPV16 E7 proteins in CaSki and C-33A (an HPV-null cervical cancer cell line) upon downregulation of USP7 using siRNA targeting specifically against human USP7. FIG. 1D. The immunoblots showing the protein level of HPV16 E7 treated with different concentrations of HBX 19818. CaSki cells were treated with different concentrations of HBX 19818 or DMSO as indicated. Total cell lysates were subjected to western blotting using USP7, β-actin and HPV16 E7 specific antibodies. FIG. 1E. The bar chart showing the relative protein level of HPV16 E7 treated with different concentrations of HBX 19818 normalized by β-actin (n=3). Data are expressed as mean±SEM. FIG. 1F. The curve chart showing the level of E7 protein with increasing concentrations of HBX 19818 treatment. The percentage of HPV16 E7 protein level was analyzed using the non-linear regression function in GraphPad™ Prism 9. Error bars show standard errors of the mean (SEM). *: P<0.05; **: P<***: P<0.001.

FIGS. 2A-2C HBX 19818 treatment destabilized the steady-state level of HPV16 E7. FIG. 2A. Immunoblots showing the protein levels of HPV16 E7, endogenous ubiquitin and USP7. CaSki cells were either transfected with expression plasmids pcDNA 3.1 (left panel) or pcDNA:Flag-USP7, treated with 3 μM of HBX 19818. After 24 hr, the cells were treated with 10 μg/ml cycloheximide at different durations, as indicated. As a control, the cells were also treated with 5 μg/ml MG132 (a proteasome inhibitor) for 2 hrs prior to adding cycloheximide. The cells were harvested and subjected to western blotting using the respective specific antibodies as indicated. Beta-actin (β-actin) was included as a loading control. CHX: cycloheximide; KDa: kilodalton. FIG. 2B. The curves showing the level of HPV16 E7 protein when co-overexpressed with USP7 or treated with HBX 19818 at different time points. The stability of HPV16 E7 was analyzed using the one-phase exponential decay function in GraphPad™ Prism 9. Error bars indicate standard error of the mean (SEM). FIG. 2C. The bar chart comparing the relative protein level of ubiquitin normalized by β-actin (n=3). Data are expressed as mean±SEM. ns: not significant, P>0.05; *: P<0.05.

FIGS. 3A-3B HBX 19818 inhibited proliferation of CaSki cells, but not SCC25 cells. The growth curves showing the cell proliferation of (FIG. 3A) CaSki (HPV16-positive) and (FIG. 3B) SCC25 (HPV-negative) cancer cells in the presence or absence of HBX 19818. Cell proliferation of CaSki was assessed using cell counting kit 8 (CCK8) assay. Cells were seeded into 96-well plates and incubated with either DMSO (vehicle control) or HBX 19818 at 3 μM or 6 μM for different days as indicated. Optical density (OD) value at 450 nm indicating relative proliferation was measured daily (n=3). Error bars indicate standard error of the mean (SEM).

FIGS. 4A-4D HBX 19818 inhibited transformation of CaSki cells, but not SCC25 cells. The representative images showing the whole-well view and morphology of colonies formed by (FIG. 4A) CaSki (HPV16-positive) and (FIG. 4C) SCC25 (HPV-negative) cancer cells. Cells were seeded into 0.35% agarose gel containing DMEM with 10% FBS, and grew for 14 days. The agarose gel was stained with crystal violet, and images were taken using a digital camera. The colony morphology was observed using an optical microscope at 100× magnification. The bar chart comparing the number of colonies of (FIG. 4B) CaSki or (FIG. 4D) SCC25 cells with DMSO or HBX 19818 at concentrations indicated (n=3). Data indicated are mean±standard error of the mean (SEM). *: P<0.05.

FIGS. 5A-5D HBX 19818 treatment inhibited migration of CaSki cells, but not SCC25 cells. The representative images showing the cell migration of (FIG. 5A) CaSki (HPV16-positive) and (FIG. 5C) SCC25 (HPV-negative) cancer cells after scratching. Cells were seeded into 12-well plate and scratched when reached 100% confluence, followed by incubation with DMSO (vehicle control) or HBX 19818 at concentrations indicated for 24 hours. Cell migration was observed and recorded using a time-lapse microscope every 6 hours. The bar chart comparing the relative coverage areas of (FIG. 5B) CaSki and (FIG. 5D) SCC25 with DMSO or HBX 19818 (n=3). Data indicated are mean±SEM. *: P<0.05; **: P<0.01.

FIGS. 6A-6D. HBX 19818 treatment inhibited invasion of CaSki cells, but not SCC25 cells. The representative images showing the cell invasion of (FIG. 6A) CaSki (HPV-positive) and (FIG. 6C) SCC25 (HPV-negative) cancer cells in the presence of DMSO (vehicle control) or HBX 19818 at concentrations indicated. Cells were seeded into the invasion chamber with plain medium, followed by treatment with either DMSO or HBX 19818 for 24 hours. Cell invasion was observed and recorded using an optical microscope at 40× magnification. The bar graph comparing the relative cell invasion ability of (FIG. 6B) CaSki and (FIG. 6D) SCC25 with DMSO or HBX 19818 (n=3). Data indicated are mean±standard error of the mean (SEM). **: P<0.01; ***: P<0.001.

### BRIEF DESCRIPTION OF THE SEQUENCES

SEQ ID NO: 1: Target Sequence of siRNA used as a deubiquitinase inhibitor

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” is used provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

“Treatment”, “treating”, “palliating” and “ameliorating” (and grammatical variants of these terms), as used herein, are used interchangeably. These terms refer to an approach for obtaining beneficial or desired results including but not limited to therapeutic benefit. A therapeutic benefit is achieved with the eradication or amelioration of one or more of the physiological symptoms associated with the underlying cancer such that an improvement is observed in the patient, notwithstanding that the patient may still be afflicted with the cancer.

As used herein, the term “cancer” refers to the presence of cells possessing abnormal growth characteristics, such as uncontrolled proliferation, immortality, metastatic potential, rapid growth and proliferation rate, perturbed oncogenic signaling, and certain characteristic morphological features.

The term “effective amount” or “therapeutically effective amount” refers to that amount of an inhibitor described herein that is sufficient to affect the intended application, including but not limited to disease treatment. The therapeutically effective amount may vary depending on the intended application (in vitro or in vivo) or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

In some embodiments of the invention, the method comprises administration of multiple doses of the compositions of the subject invention. The method may comprise administration of 1, 2, 3, 4, 5, 6, 7, 8, 9, 10, 15, 20, 25, 30, 35, 40, or more therapeutically effective doses of a composition of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. Moreover, treatment of a subject with a therapeutically effective amount of the compositions of the invention can include a single treatment or can include a series of treatments. It will also be appreciated that the effective dosage of a composition used for treatment may increase or decrease over the course of a particular treatment. Changes in dosage may result and become apparent from the results of diagnostic assays or imaging techniques for detecting tumor sizes known in the art. In some embodiments of the invention, the method comprises administration of the composition several time per day, including but not limiting to 2 times per day, 3 times per day, and 4 times per day.

As used herein, the term “subject” refers to an animal, needing or desiring delivery of the benefits provided by a therapeutic composition. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of enamel, an organ, tissue, or system in the body. The subject can be of any age or stage of development, including infant, toddler, adolescent, teenager, adult, or senior. The terms “subject” and “patient” can be used interchangeably.

The terms “antagonist” and “inhibitor” may be used interchangeably, and they refer to a compound having the ability to inhibit a biological function of a target protein, whether by inhibiting the activity or expression of the target protein. Accordingly, the terms “antagonist” and “inhibitor” are defined in the context of the biological role of the target protein.

Cancers suitable for treatment according to the disclosed methods include, for example, HPV-associated cancers, such as, for example, cancers of the head and neck, cancers of the uterine cervix, oropharynx, tonsils, base of tongue, anus, rectum, vulva, vagina, penis, and skin cancers, such as basal cell carcinoma, squamous cell carcinoma, and melanoma.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Compositions

In certain embodiments, the compositions and methods according to the subject invention utilize at least one deubiquitinase inhibitor. In preferred embodiments, the deubiquitinase inhibitor is a ubiquitin specific protease 7 (USP7) inhibitor, such as, for example, HBX 19818, and/or a small interfering RNA (siRNA) that can target USP7. In preferred embodiments, the siRNA downregulates USP7. In certain embodiments, the downregulation of USP7 downregulates the HPV16 E7 protein.

A deubiquitinase inhibitor may be added to compositions at concentrations of about 0.0001 to about 50% by weight (wt %), preferably about 1 to about 10 wt %, and most preferably about 3% to about 5 wt %. In another embodiment, a deubiquitinase inhibitor can be in combination with an acceptable carrier and/or excipient, in that a deubiquitinase inhibitor may be presented at concentrations of about 0.0001 to about 50% (v/v), preferably, about 1 to about 10% (v/v), more preferably, about 3 to about 3% (v/v). In certain embodiments, a deubiquitinase inhibitor may be added to compositions at concentrations of about 0.01 μM to about 100 μM, 0.1 μM to about 100 μM, about 1 μM to about 10 μM, about 3 μM to about 6 μM, about 3 μM, about 0.133 μM, or about 6 μM.

In certain embodiments, the USP7 inhibitor can be a siRNA. In certain embodiments, the siRNA can downregulate USP7. In certain embodiments, the siRNA can target the following sequence:

TACGTGACTTGCTCCCAGTTATGTGTGACAGAGCAGGATTTATTCAAGATACTAGCC TTATCCTCTATGAGGAAGTTAAACCGAATTTAACAGAGAGAATTCAGGACTATGACG TGTCTCTTGATAAAGCCCTTGATGAACTAATGGATGGTGACATCATAGTATTTCAGA AGGATGACCCTGAAAATGATAACAGTGAATTACCCACCGCAAAGGAGTATTTCCGA GATCTCTACCACCGCGTTGATGTCATTTTCTGTGATAAAACAATCCCTAATGATCCTG GATTTGTGGTTACGTTATCAAATAGAATGAATTATTTTCAGGTTGCAAAGACAGTTG CACAGAGGCTCAACACAGATCCAATGTTGCTGCAGTTTTTCAAGTCTCAAGGTTATA GGGATGGCCCAGGT (SEQ ID NO: 1). In certain embodiments, the siRNA is Mission® esiRNA (Cat No. EHU131171, Sigma-Aldrich, Woodlands, TX). In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, intracerebroventricularly or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

The compositions used for the treatment can include, for example, DMEM medium (about mM glycine, about 0.4 mM L-arginine hydrochloride, about 0.2 mM L-cystine 2HCl, about mM L-histidine hydrochloride-H₂O, about 0.8 mM L-isoleucine, about 0.8 mM L-leucine, about 0.8 mM L-lysine hydrochloride, about 0.2 mM L-methionine, about 0.4 mM L-Phenylalanine, about 0.4 mM L-serine, about 0.8 mM L-threonine, about 0.08 mM L-tryptophan, about 0.4 mM L-tyro sine disodium salt dihydrate, about 0.8 mM L-valine, about 0.03 mM choline chloride, about 0.008 mM D-calcium panthothenate, about 0.009 mM folic acid, about 0.03 mM niacinamide, about 0.02 mM pyridoxine hydrochloride, about 0.001 mM riboflavin, about 0.011 thiamine hydrochloride, about 0.04 mM i-Inositol, about 1.8 mM calcium chloride, about 0.5 mM ferric nitrate, about 0.8 nN magnesium sulfate, about 5.3 mM potassium chloride, about 44 mM sodium bicarbonate, about 110 mM sodium chloride, about 0.9 mM sodium phosphate monobasic, about 25 mM D-glucose (dextrose), about 0.04 mM phenol red, about 1 mM sodium pyruvate), about 2 mM L-glutamine, about 10% fetal bovine serum, less than about 1% DMSO, or any combination thereof.

In one embodiment, the compositions of the subject invention can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

### Methods of Using the Subject Compositions

In certain embodiments, a deubiquitinase inhibitor can be administered to a subject. Any means of administration that can permit a deubiquitinase inhibitor to contact cells in a subject, including, for example, orally, intravenously, intraperitoneally, intramuscularly, intrathecally, or subcutaneously are envisioned in the subject methods.

In certain embodiments, a deubiquitinase inhibitor can contact healthy cells of subject and/or tumor cells or cancerous cells, including, for example, cancer cell lines. In certain embodiments, a deubiquitinase inhibitor can contact cells in the uterine cervix, oropharynx, tonsil, base of tongue, anus, rectum, vulva, vagina, penis, including cancerous cells of the uterine cervix, oropharynx, tonsil, base of tongue, anus, rectum, vulva, vagina, or penis. In certain embodiments, a deubiquitinase inhibitor, such as, for example, HBX 19818 and/or an siRNA that can target USP7, can inhibit cervical cancer, oropharyngeal cancer, tonsil cancer, tongue cancer, anal cancer, rectal cancer, vulvar cancer, vaginal cancer, penile cancer, and other solid tumors.

In certain embodiments, the deubiquitinase inhibitor can bind to protein in cells or on the surface of cells of a subject, including, for example, cancerous cells, such as, for example, cancerous cells caused by HPV. In certain embodiments, the deubiquitinase inhibitor can inhibit the growth of tumor cells. The deubiquitinase inhibitor can inhibit the growth of tumor cells by modulating the HPV E7 protein level and/or the function of HPV E7, including, for example, the cancer promoting role of HPV E7. In certain embodiments, HPV E7 can form a complex with USP7, which is a deubiquitinating enzyme of host cells. This binding brings about an increased E7 steady-state level. In certain embodiments, compositions of the subject invention can inhibit the HPV E7 and USP7 complex, which can decrease the HPV E7 protein steady-state level. In certain embodiments, administration of compositions of the subject invention can result in the destabilization of HPV E7, resulting in suppression of HPV E7 protein level by about 5% to about 75%, about 10% to about 60%, preferably, about 20% to about 50%, about 30% to about 50%, about 40% to about 50%, or about 50%. In certain embodiments, administration of compositions of the subject invention can result in the suppression of a deubiquitinase protein (e.g., USP7) level by about 5% to about 75%, about 10% to about 60%, preferably, about 20% to about 50%, about 30% to about 50%, about 40% to about 50%, or about 50%. In certain embodiments, the composition of the subject invention can inhibit of the ability of HPV-positive cancer cells to proliferate, grow in an anchorage-independent manner, migrate, and invade through the basal membrane, specifically in HPV-containing cancer cells and not in HPV-null cancer cells.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Identification of HBX 19818 as the Potent HPV Inhibitor

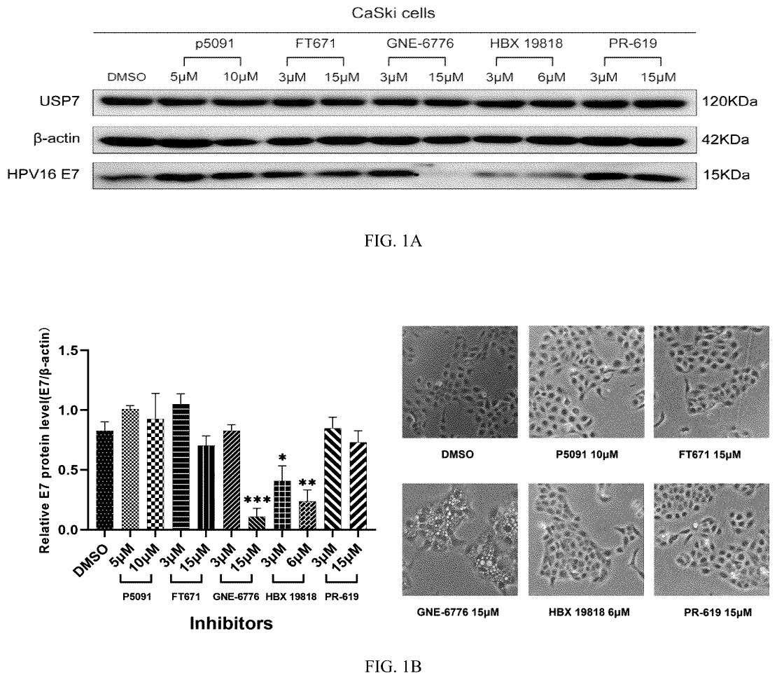

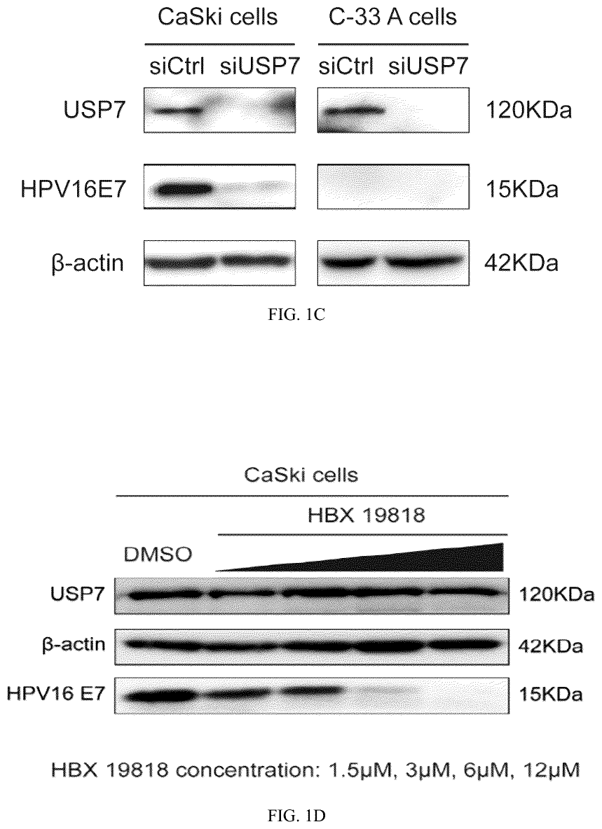

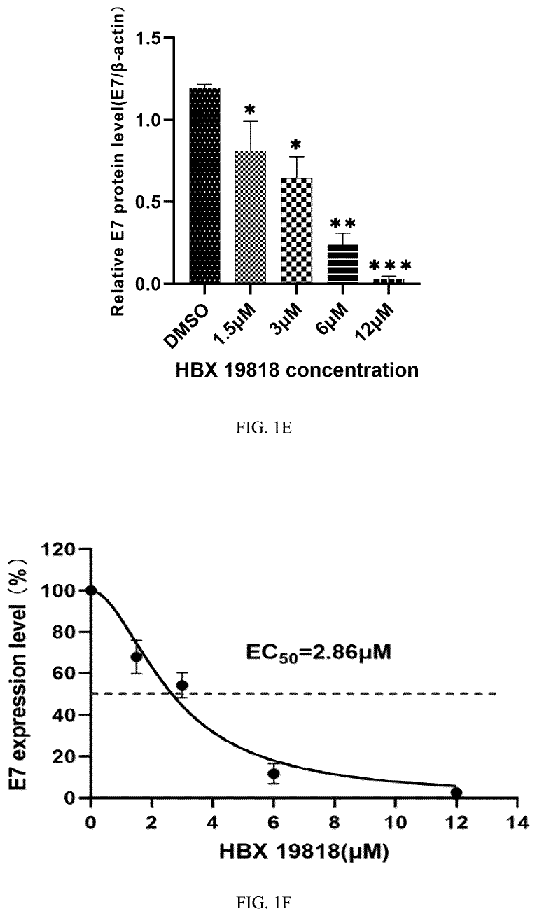

Firstly, we screened four USP7-specific inhibitors (P5091, FT671. GNE-6776, and HBX 19818) and 1 pan-USPs inhibitor (PR-619) to characterize their inhibitory effect on E7 expression in HPV-containing cancer cells. We treated CaSki (HPV16-positive) cancer cells with P5091 at 5 μM and 10 μM; FT671, GNE-6776 and PR-619 at 3 μM and 15 μM; as well as HBX 19818 at 3 μM and 6 μM. These concentrations were chosen based on the reported IC₅₀ values. After 24-hr treatment, the cells were collected, and total cell lysates were resolved by SDS-PAGE. The expression levels of USP7 and E7 were detected using respective specific antibodies. From triplicate independent experiments, we observed that HBX 19818 treatment resulted in a dramatic reduction of E7 protein level, and was at the greatest extent compared to other inhibitors (FIGS. 1A and 1B). Even though GNE-6776 treatment at 15 μM decreased the E7 protein level drastically, intracellular vacuoles indicating toxicity were found in treated cells (FIG. 1B). To further elucidate the positive relationship between HPV16E7 and USP7, we also downregulated USP7 in C-33 A, and CaSki cells using specific siRNA against USP7. When USP7 was downregulated by siRNA, a reduced HPV16E7 protein level was observed in CaSki cells, compared with CaSki treated with siCtrl. This result indicates that HPV16E7 protein level corresponds positively with USP7 protein level (FIG. 1C). In addition, the level of E7 in CaSki cells, which are HPV16-positive cells, treated with an increasing concentration of HBX 19818 (1.5 μM, 3.0 μM, 6.0 μM, and 12.0 μM) was decreased at a dose-dependent manner (FIGS. 1D and 1E). We found that 2.8 μM of HBX 19818 was sufficient to halve the level of E7 protein in CaSki cells (FIG. 1F).

### Example 2—HBX 19818 Reduced the Stead-State Level of HPV E7

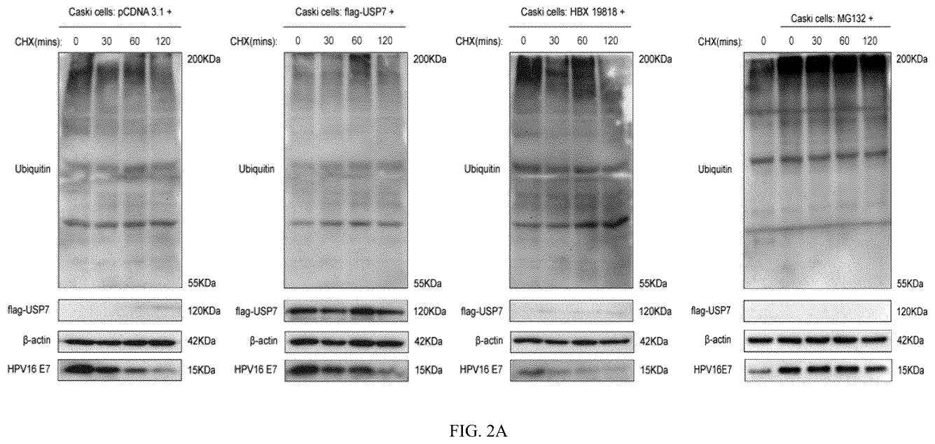

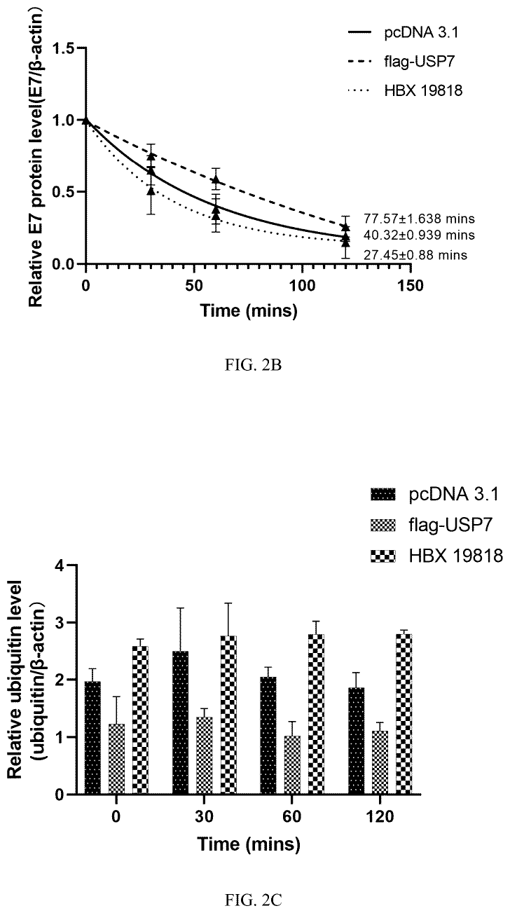

We next wanted to assess whether HBX 19818 can reverse USP7-mediated deubiquitination of E7. A cycloheximide chase assay was performed to evaluate the stability of E7 in the presence or absence of HBX 19818 treatment. CaSki cells were transfected with pcDNA3.1 (mock transfection control) or pcDNA3.1:Flag-USP7 that encoded for Flag-tagged USP7. The transfected cells were treated with 3 μM of HBX 19818 or DMSO (vehicle control) for 24 hrs. As a control, we also treated the cells with 5 μg/ml MG132 (a proteasome inhibitor known to stabilize expression of HPV16 E7) 2 hrs prior to cycloheximide treatment. Following this, the cells were treated with 10 μg/ml cycloheximide at different time points. Cells were collected, lysed, and resolved via SDS-PAGE and then subjected to western blotting. The expression levels of Flag-USP7, endogenous ubiquitin, and E7 were detected using anti-Flag-, ubiquitin- and HPV16 E7-specific antibodies, respectively (FIG. 2A). The half-life of E7 increased by almost doubled when co-expressed with USP7 (77.57±1.64 mins) compared with when E7 was expressed alone (40.32±0.94 mins). When treated with HBX 19818, the half-life of E7 was 27.45±0.88 minutes, which was only half of that of E7 alone (FIG. 2B). In addition, we also observed an increased level of ubiquitin in CaSki cells upon HBX 19818 treatment, independent of the cycloheximide-mediated halt of protein synthesis (FIG. 2C).

### Example 3—Selective Inhibitory Effect of HBX 19818 on HPV-Positive Cancer Cells

We assessed the efficacy of HBX 19818 in inhibiting cancer phenotypes of HPV-positive cancer cells (CaSki) compared to HPV-null cancer cells (SCC25, a human tongue epithelial cancer cell line). In particular, we investigated whether HBX 19818 treatment can hamper E7-mediated carcinogenic events of these cells including cell proliferation, transformation, migration, and invasion of these cells.

### Example 4—HBX 19818 Treatment Reduced Proliferation of HPV-Positive Cancer Cells, but not HPV-Null Cancer Cells

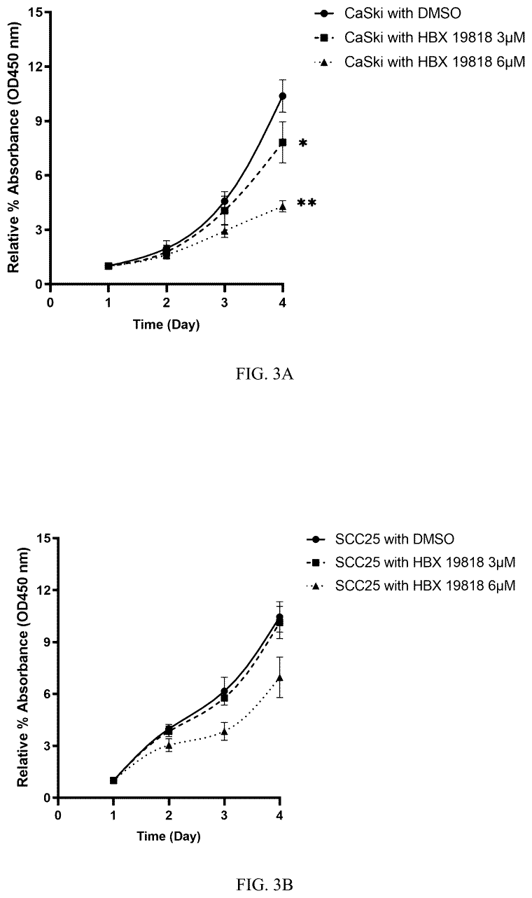

We assessed the ability of CaSki (HPV16 positive cells) and SCC25, which are HPV-null cells, to proliferate using a cell counting kit-8 (CCK8) assay. This assay measures the ability of viable cells to metabolize soluble tetrazolium salt (WST-8) to orange formazan, in which the latter can be measured at optical density (OD) of 450 nM. A day prior to drug treatment, CaSki and SCC25 were seeded into each well of 96-well plates at a density of 2000 cells/well in 100 μL of DMEM containing 10% FBS. The cells were treated with either DMSO (vehicle control) or HBX 19818 at 3 μM and 6 μM for 24, 72, and 96 hrs. CCK8 solution was mixed with cell medium at a 1:10 ratio, followed by a 1-hr incubation at 37° C. The OD value of the medium at 450 nm was measured using LEDETECT 96 microplate reader (Labexim Products). We observed that when compared to vehicle control (DMSO) (10.38±0.90 folds), treatment of CaSki with 6 μM of HBX 19818 exhibited the lowest cell proliferation ability (4.29±0.31 folds, P<0.001), followed by CaSki with 3 μM HBX 19818 (7.82±1.14 folds, P<0.01) (FIG. 3A). In SCC25 cells, 6 μM of HBX 19818 inhibited cell proliferation slightly (7.29±1.24 folds) while 3 μM of HBX 19818 had a comparable proliferation ability (10.12±0.94 folds) as the SCC25 cells treated with DMSO (10.45±0.88 folds) (FIG. 3B).

### Example 5—HBX 19818 Treatment Inhibited Transformation of HPV-Positive Cancer Cells, but not HPV-Null Cancer Cells

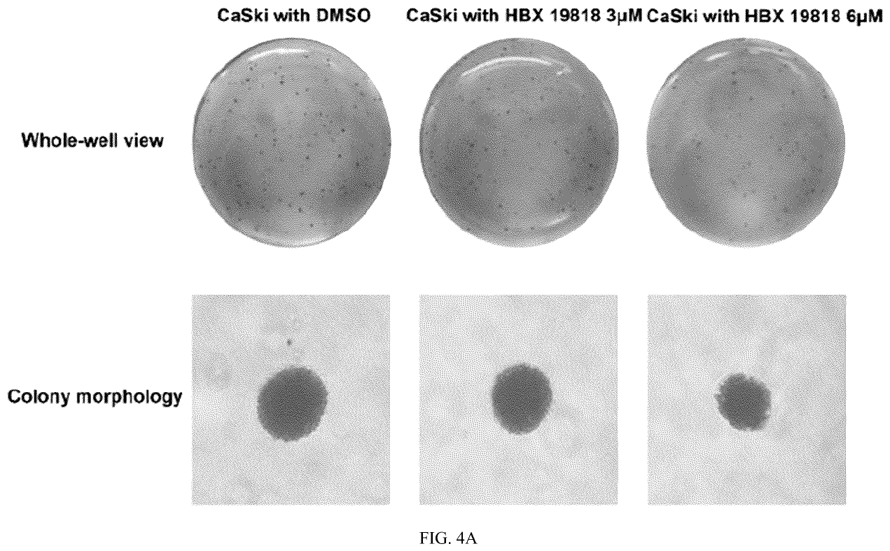

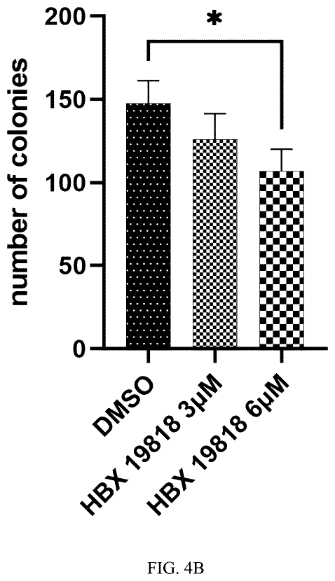

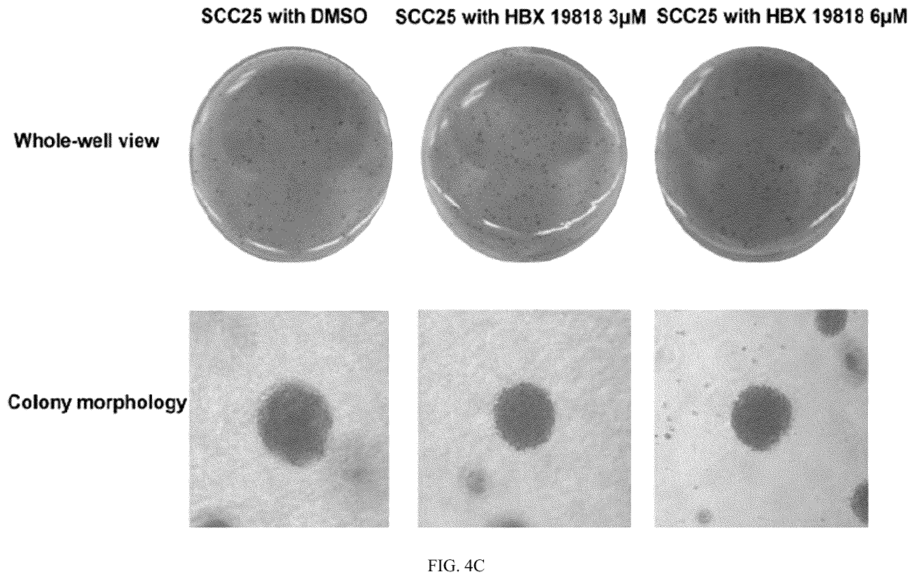

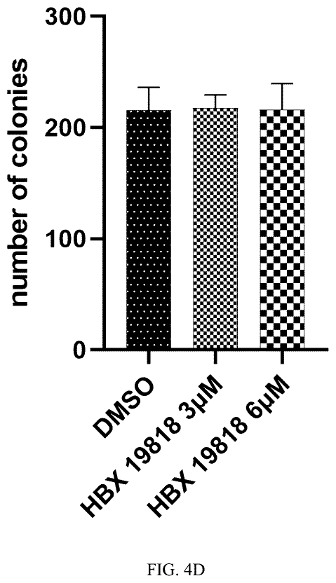

We performed a soft agar colony formation assay to assess the ability of CaSki and SCC25 to grow in an anchorage-independent manner, in the presence or absence of HBX 19818 treatment. CaSki and SCC25 cells were seeded into 0.7% soft agar containing 2×DMEM and 20% FBS. The cells were incubated at 37° C. for 14 days. After a 2-week incubation, the agarose gel was stained with crystal violet. Colonies formed were counted and measured. We observed that CaSki cells treated with DMSO formed a dramatically higher number of colonies (88.17±5.92) than those treated with 6 μM of HBX 19818 (62.67±2.03) (FIGS. 4A and 4B). The number of colonies formed by CaSki cells treated with 3 μM of HBX 19818 (74.67±7.99) was also higher than that of CaSki cells treated with DMSO. In contrast, there was no difference in the number of colonies formed by SCC25 cells, regardless of treatment with DMSO or HBX 19818 (FIGS. 4C and 4D).

### Example 6—HBX 19818 Treatment Reduced Migration Ability of HPV-Positive Cancer Cells, but not HPV-Null Cancer Cells

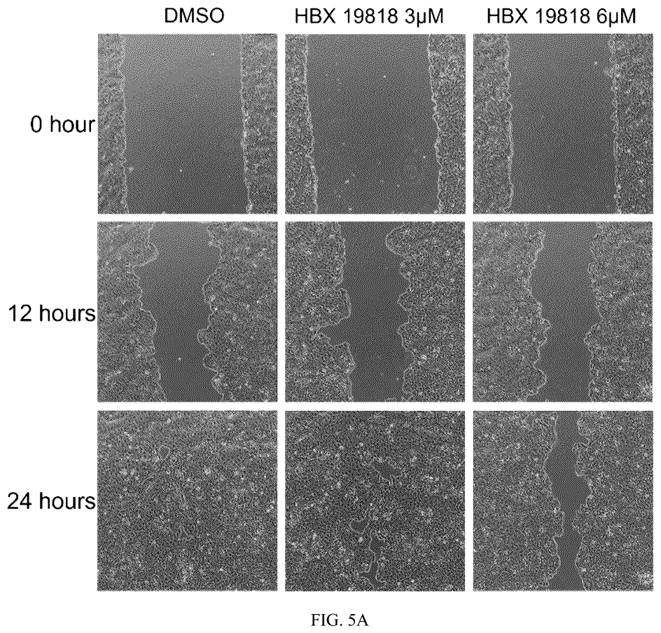

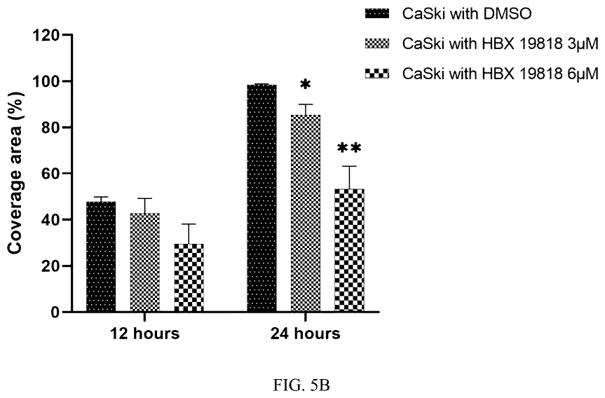

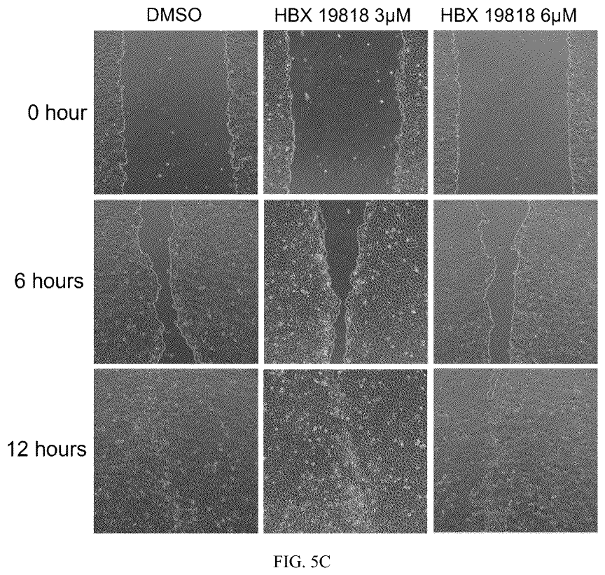

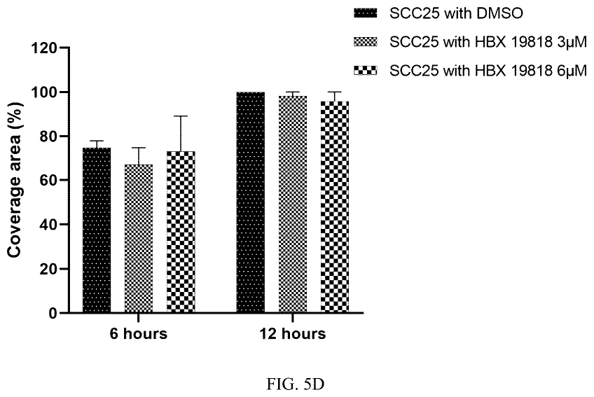

We performed a wound-healing assay to ascertain the ability of CaSki and SCC25 cells to migrate under HBX 19818 treatment. Cells were seeded into a 12-well plate and cultured to reach 100% confluency. Cells were then scratched to make artificial wounds on the monolayer of cells and incubated with either DMSO or HBX 19818 at 3 μM or 6 μM. Cells migration was monitored using time-lapse microscopy where images were taken 6-hourly for a duration of 24 hrs. We observed that CaSki cells incubated with DMSO demonstrated a significant increase in cell migration ability (98.46%±0.36%) compared with CaSki cells treat with 3 μM of HBX 19818 (85.58%±4.39%, p<0.01) and 6 μM of HBX 19818 (53.24%±8.96%, p<0.001) (FIGS. 5A and 5B). However, no differences were observed in SCC25 cells treated with DMSO or HBX 19818 (FIGS. 5C and 5D).

### Example 7—HBX 19818 Treatment Inhibited the Invasive Ability of HPV-Positive Cancer Cells, but not HPV-Null Cancer Cells

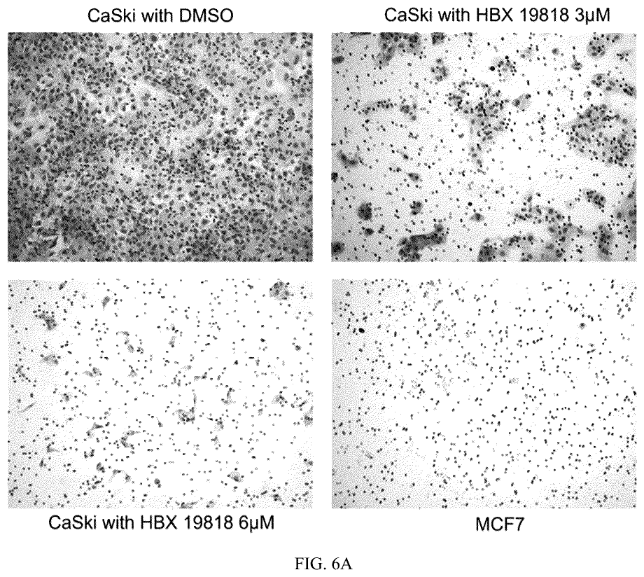

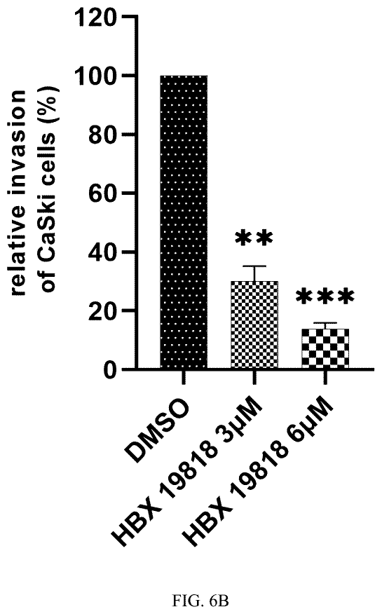

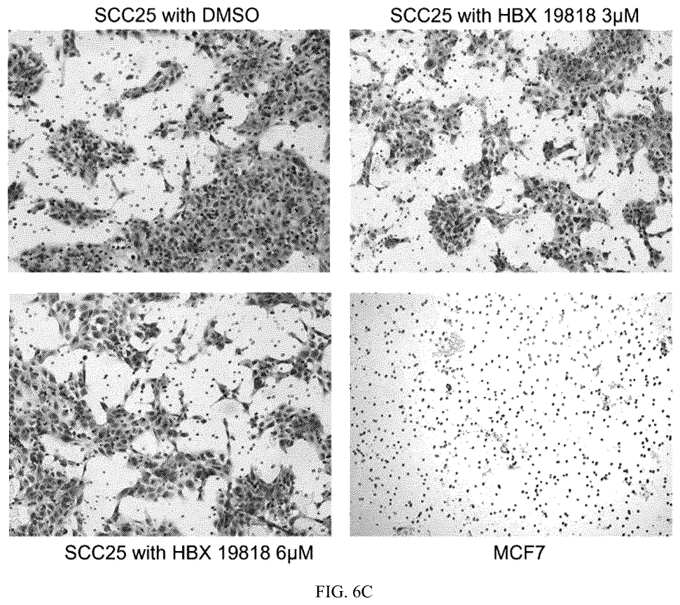

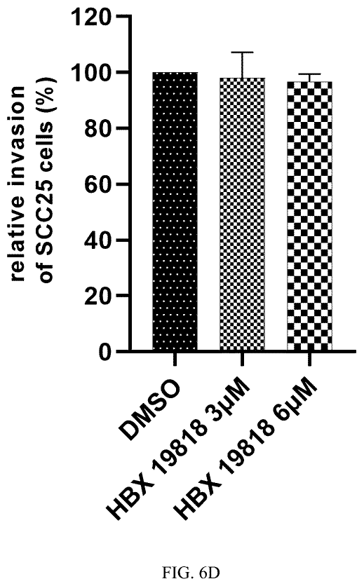

We next examined the ability of CaSki and SCC25 cells to invade through Matrigel, which mimics the basement membrane. Approximately 5×10⁴ of CaSki and SCC25 cells were seeded into the inserts containing serum-free DMEM medium with either DMSO or HBX 19818. We included MCF7, a non-invasive breast cancer cell line as a negative control. DMEM supplemented with 20% FBS was added into the bottom chamber to attract cells to migrate from the upper to the lower surface of the inserts. After 24-hr incubation at 37° C., the non-invasive cells on the upper surface were removed using cotton swabs. Cells on the lower surface were fixed and stained using hematoxylin. From microscopic images, we observed that CaSki treated with DMSO (100%) had a greatest invasive ability in comparison to CaSki cells treated with 3 μM of HBX 19818 (30%±2.97%, p<0.01), and 6 μM HBX 19818 (13.85%±1.21%, p<0.001) (FIGS. 6A and 6B). Similarly, we observed that the invasive ability of SCC25 was similar, regardless the cells were treated with DMSO or HBX 19818 (FIGS. 6C and 6D).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method of treating a cancer comprising: administering a composition comprising a deubiquitinase inhibitor to a subject, wherein the cancer is caused by a human papillomavirus (HPV), is a cancer of the anogenital region, or is a cancer of the head or neck region, or is a skin cancer.

2. The method of claim 1, wherein the cancer of the anogenital region is uterine, cervix, anus, rectum, vulva, vagina, penis, or any combination thereof.

3. The method of claim 1, wherein the cancer of the head and neck region is oropharynx, tonsil, base of tongue, or any combination thereof.

4. The method of claim 1, wherein the skin cancer is basal cell carcinoma, squamous cell carcinoma, or melanoma.

5. The method of claim 1, wherein the deubiquitinase inhibitor is a USP7 inhibitor.

6. The method of claim 5, wherein the USP7 inhibitor is HBX 19818.

7. The method of claim 1, wherein the deubiquitinase inhibitor decreases a level of an HPV E7 protein in the subject after administration of the composition.

8. The method of claim 7, wherein the level of the HPV E7 protein is reduced by at least 10%, 20%, 30%, 40%, 50%, or 60% in the subject.

9. The method of claim 1, further comprising administering an siRNA to the subject.

10. The method of claim 9, wherein the siRNA targets
(SEQ ID NO: 1)
TACGTGACTTGCTCCCAGTTATGTGTGACAGAGCAGGATTTATTCAAGA
TACTAGCCTTATCCTCTATGAGGAAGTTAAACCGAATTTAACAGAGAGA
ATTCAGGACTATGACGTGTCTCTTGATAAAGCCCTTGATGAACTAATGG
ATGGTGACATCATAGTATTTCAGAAGGATGACCCTGAAAATGATAACAG
TGAATTACCCACCGCAAAGGAGTATTTCCGAGATCTCTACCACCGCGTT
GATGTCATTTTCTGTGATAAAACAATCCCTAATGATCCTGGATTTGTGG
TTACGTTATCAAATAGAATGAATTATTTTCAGGTTGCAAAGACAGTTGC
ACAGAGGCTCAACACAGATCCAATGTTGCTGCAGTTTTTCAAGTCTCAA
GGTTATAGGGATGGCCCAGGT.

11. The method of claim 9, wherein the siRNA downregulates USP7 expression.

12. The method of claim 11, wherein the downregulation of USP7 downregulates an HPVE7 protein.

13. The method of claim 1, wherein the deubiquitinase inhibitor inhibits the ability of HPV-positive cancer cells to proliferate, transform, migrate, invade through the basement membrane, or any combination thereof.

14. The method of claim 1, wherein the USP7 inhibitor is at a concentration of about 1 μM to about 10 μM in the composition.

15. The method of claim 1, wherein the composition further comprises at least one carrier or excipient.
